(19) United States
(12) Patent Application Publication
(10) Pub. No.: US 2005/0277139 A1
(43) Pub. Date: Dec. 15, 2005

(54) Methods and apparatus for the detection and validation of microRNAs

(76) Inventors: Itzhak Bentwich, Kvutzat Shiler (IL); Amir Avniel, Givatayim (IL); Yael Karov, Tel Aviv (IL); Ranit Aharonov, Tel Aviv (IL)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 11/114,879

(22) Filed: Apr. 26, 2005

Related U.S. Application Data
(60) Provisional application No. 60/521,433, filed on Apr. 26, 2004.
(60) Provisional application No. 60/521,563, filed on May 25, 2004.
(60) Provisional application No. 60/522,300, filed on Sep. 14, 2004.
(60) Provisional application No. 60/522,454, filed on Oct. 3, 2004.
(60) Provisional application No. 60/522,453, filed on Oct. 3, 2004.
(60) Provisional application No. 60/522,762, filed on Nov. 4, 2004.

(51) Int. Cl. C12Q 1/68; C07H 21/02; C12P 19/34

(52) U.S. Cl. 435/6; 536/25.4; 435/91.2

(57) ABSTRACT

The present invention provides methods and apparatus for detecting, sequencing, cloning and otherwise validating nucleic acids, including microRNAs. The methods make use of a plurality of sequence-specific recognition reagents which can also be used for classification of biological samples, and to characterize the expression of certain microRNAs.

### CROSS REFERENCE TO RELATED APPLICATIONS

The present application claims the benefit of U.S. Provisional Patent Application No. 60/521,433, filed Apr. 26, 2004, U.S. Provisional Patent Application No. 60/521,563, filed May 25, 2004, U.S. Provisional Patent Application No. 60/522,300, filed Sep. 14, 2004, U.S. Provisional Patent Application No. 60/522,454, filed Oct. 3, 2004, U.S. Provisional Patent Application No. 60/522,453, filed Oct. 3, 2004 and U.S. Provisional Patent Application No. 60/522,762, filed Nov. 4, 2004.

### FIELD OF THE INVENTION

The present invention is related to methods for identifying nucleic acids, such as microRNAs.

### BACKGROUND OF THE INVENTION

MicroRNAs (miRNAs) are short RNA oligonucleotides of approximately 22 nucleotides that play an important role in gene regulation. miRNAs regulate gene expression by targeting mRNAs for cleavage or translational repression. Although miRNAs are present in a wide range of species including C. elegans, Drosophilla and humans, they have only recently been identified. Although a limited number of miRNAs have been identified by extracting large quantities of RNA, miRNAs are difficult to identify using standard methodologies as a result of their small size.

Computational approaches have recently been developed to identify the remainder of miRNAs in the genome. Tools such as MiRscan, MiRseeker and those described in U.S. Patent Application No. 60/522,459, Ser. Nos. 10/709,577 and 10/709,572 have predicted a large number of miRNAs in the human genome. It would be beneficial to validate those predicted miRNAs that exist in vivo, and to determine the expression profiles of these miRNAs.

Microarrays allow the high throughput analysis of gene expression. Microarray technology is based on measuring the hybridization of a target sequence to a probe sequence attached to a substrate. A limitation of microarrays is that only hybridization is measured, without any indication of the degree of complementarity between the probe and the target gene. This indirect evidence of a target sequence is of little concern when the target sequence is a relatively long sequence that can be positively identified by using multiple probe sequences per target. Such a practice is of little benefit for the identification and confirmation of short nucleic acid sequences, such as miRNAs.

### SUMMARY OF THE INVENTION

The present invention is related to a method of detecting a miRNA. An array may be provided comprising a solid substrate and a plurality of positionally distinguishable polynucleotides attached to the solid substrate. Each polynucleotide may comprise a miRNA. The array may be contacted with a plurality of target polynucleotides comprising a complement of a miRNA under conditions permitting hybridization. Hybridization of a target sequence to the miRNA may be detected A miRNA may be detected when hybridization is above background.

The plurality of target polynucleotides may be produced by providing RNA comprising a plurality of miRNA. The RNA may be less than 160 nucleotides in length. Adapters may then be ligated to the 5′ and 3′ ends of the RNA. The adapters may comprise a restriction site, which may be used later to remove the adapters. The adapters may be DNA-RNA hybrids. First strand cDNA of the 5′-adapter-miRNA-adapter-3′ may then be prepared. The adapter-miRNA-adapter may then be amplified. cRNA may then be prepared using a promoter complementary to the 3′ adapter.

A miRNA may also be detected by providing a plurality of target polynucleotides comprising a miRNA, a labeled oligo that is complementary to a portion of the target nucleotides, and substrate comprising a capture oligonucleotide comprising at least 16 nucleotides of a miRNA complementary sequence. The target nucleotides may then be contacted with the labeled oligo and substrate. Hybridization of the target nucleotides, labeled oligo and substrate may then be detected.

The present invention is related to a method of isolating a miRNA. A solid substrate may be provided comprising a capture oligonucleotide comprising at least 16 nucleotides of a miRNA sequence. The capture oligonucleotide may be contacted with a plurality of target polynucleotides comprising a complement of a miRNA under conditions permitting hybridization. The target polynucleotides may then be eluted from the capture oligonucleotide. The eluted target polynucleotide may be sequenced. The eluted target polynucleotide may also be sequenced.

### BRIEF DESCRIPTION OF THE DRAWINGS

FIG. 1 demonstrates model for maturation of mammalian miRNAs.

FIG. 2 demonstrates the preparation of a target cDNA library.

FIG. 3 shows results using a MIRChip. Panel A shows the signal ratio on a MIRChip using probes that include a miRNA precursor, a miRNA in the 5′ portion of a probe, and a miRNA precursor with no more than 16 nucleotides of the miRNA sequence. Panel B shows a mismatch analysis of pre-miRNA-125b showing the location on the WT sequence of the mismatches that were included.

FIG. 4 shows results using different types of probes. Panel A shows results of probes containing miRNAs in the 5′, middle, or 3′ end of the probe. Panel B shows results of probes containing one, two, or three miRNA copies. Panel C shows results of probes containing two or three miRNA copies containing no mismatches, 2 mismatches in the 5′ miRNA, 2 mismatches in the middle or 3′ miRNA, or miRNA 2 mismatches in each of 2 miRNAs.

FIG. 5 shows the effect of the number of mismatches in the probe on the signal intensity of the MIRChip at either 50° C. or 60° C.

FIG. 6 shows expression of the 150 human miRNAs in five tissues and HeLa cells.

FIG. 7 shows expression of tissue-specific or highly enriched miRNAs in five human tissues using MIRChip.

FIG. 8 shows an illustration of the MIRAclone method.

FIG. 9 shows the cloning of human mir-21. Panel A shows amplification of recovered library molecules using primers matching to the adaptors. The PCR product was detected from cRNA as target (lane 1), amplified cDNA (lane 2) or non-amplified cDNA (lane 3). No PCR products were observed when the entire procedure was performed without the addition of library molecules (lane 4), or in mock PCR (lane 5). Panel B shows PCR on ligation. The presence of the candidate miRNAs in the ligated vector was verified using a primer specific to mir-21 and a primer located downstream (lanes 1-4) or upstream (lanes 5-8) of the MCS on the vector. Amplified PCR products were detected only when specific ligation was tested (lanes 1, 5 amplified cDNA, and lanes 2, 6 non-amplified cDNA). Controls of ligation from another recovered miRNA gave no amplification (lanes 3 and 7). Panel C shows colony hybridization with DIG labeled oligonucleotide probes complementary to mir-21. Panel D shows colony PCR on ten colonies with a mir-21 specific primer and a primer located upstream or downstream to the MCS of the vector. Only clones that were positive in the colony hybridization assay (lanes 1, 4, 6, 7, and 9, also marked in Panel C) gave the expected PCR products. Mock PCR gave no amplification (lane 11).

FIG. 10 shows clones containing the authentic sequence of miRNAs expressed above background levels in a microarray analysis of placenta-derived miRNAs.

FIG. 11 describes the elongation of capture oligonucleotide. The top capture oligonucleotide is composed of the longer mature mir-21 sequence. Capture oligonucleotides containing extra nucleotides derived from the precursor sequence are found below. The sequence of mir-21 is underlined.

FIG. 12 shows the structure and sequence of the six predicted hairpins and miRNAs used as templates for MIRclone cloning. The sequence composing the capture oligonucleotide is boxed. The sequence of the predicted miRNA is in bold. The arrows mark the boundary of the actual sequence of the cloned miRNAs. For mir-RG-27 and mir-RG-21, the multiple arrows mark the various 3′ ends observed in different clones.

FIG. 13 shows a chromosomal cluster analysis of novel miRNAs. The location of the novel miRNAs relative to each other or to published miRNAs is depicted for chromosomes 14, 17, and X. The boxes represent miRNA precursors and the thick line within the box represents the location of the mature miRNA sequence within the precursor.

### DETAILED DESCRIPTION

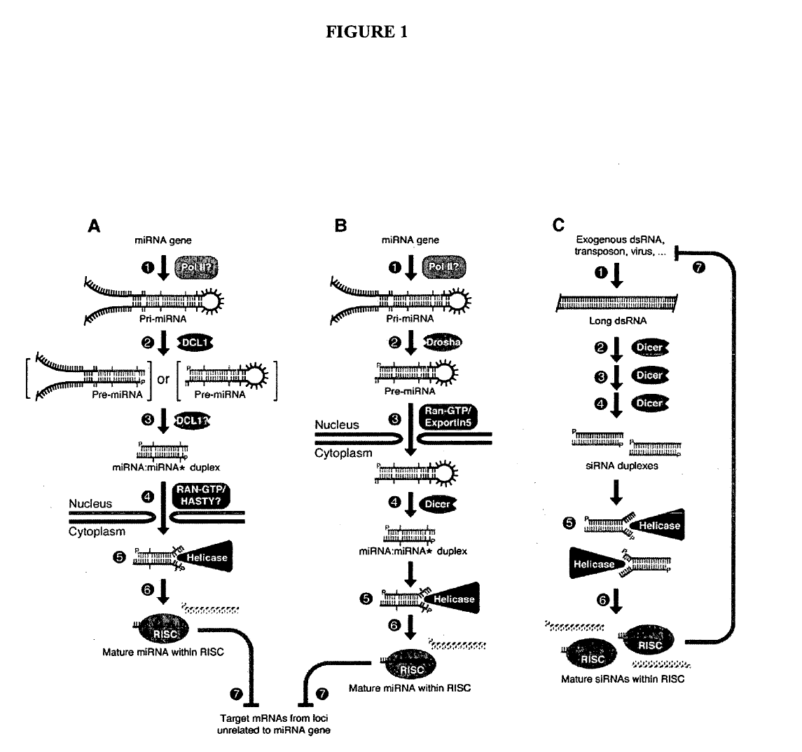

While not being bound by theory, the current model for maturation of mammalian miRNAs is shown in FIG. 1. The first step may be the nuclear cleavage of the pri-miRNA. The pri-miRNA may be part of a polycistronic RNA comprising multiple miRNAs. Cleavage of the pri-miRNA may liberate a 60-70 nt stem loop intermediate, known as the miRNA precursor, or the pre-miRNA. The processing of the pri-miRNA may be performed by the Drosha RNase III endonuclease, which may cleave both strands of the stem at sites near the base of the primary stem loop. Drosha may cleave the RNA duplex with a staggered cut typical of RNase III endonucleases, and thus the base of the pre-miRNA stem loop may have a 5′ phosphate and ˜2 nt 3′ overhang. The pre-miRNA may then be actively transported from the nucleus to the cytoplasm by Ran-GTP and the export receptor Ex-portin-5.

The cleavage by Drosha may define one end of the mature miRNA. The other end of the miRNA may be processed in the cytoplasm by the enzyme Dicer. Dicer, also an RNase III endonuclease, may also be involved in generating the small interfering RNAs (siRNAs) that mediate RNA interference (RNAi). Dicer perform may perform an activity in metazoan miRNA maturation similar to that which it performs in cleaving double-stranded RNA during RNAi. Dicer may first recognize the double-stranded portion of the pre-miRNA, perhaps with particular affinity for a 5′ phosphate and 3′ overhang at the base of the stem loop. Then, at about two helical turns away from the base of the stem loop, Dicer may cut both strands of the duplex. The cleavage by Dicer may cleave off the terminal base pairs and loop of the pre-miRNA, leaving the 5′ phosphate and 2 nt 3′ overhang characteristic of an RNase III and producing an siRNA-like imperfect duplex that comprises the mature miRNA and a similar-sized fragment derived from the opposing arm of the pre-miRNA. The fragments from the opposing arm, called the miRNA* sequences, are found in libraries of cloned miRNAs but typically at much lower frequency than the miRNAs.

The specificity of the initial cleavage mediated by Drosha may determine the correct register of cleavage within the miRNA precursor and thus may define both mature ends of the miRNA. The determinants of Drosha recognition may include a larger terminal loop (y 10 nt). From the junction of the loop and the adjacent stem, Drosha may cleave approximately two helical turns into the stem to produce the pre-miRNA. Beyond the pre-miRNA cleavage site, approximately one helical turn of stem extension (˜10 nt) may be essential for efficient processing.

Following cleavage, the miRNA pathway may be identical to the RNA silencing pathways known as posttranscriptional gene silencing. Although initially present as a double-stranded species with miRNA*, the miRNA may eventually become incorporated as single-stranded RNAs into a ribonucleoprotein complex, known as the RNA-induced silencing complex (RISC). When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target messages based on high levels of complementarity between the miRNA and the mRNA. The target sites in the mRNA may be in the 5′ UTR, the 3′ UTR or in the coding region. Interesting multiple miRNAs may regulate the same mRNA target by recognizing the same or multiple sites. The presence of multiple miRNA complementarity sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has sufficient complementarity to the miRNA. When a miRNA guides cleavage, the cut may be between the nucleotides pairing to residues 10 and 111 of the miRNA. Alternatively, the miRNA may repress translation if the miRNA does not have sufficient complementarity to the miRNA. Translational repression may be more prevalent in animals since animals may have a lower degree of complementarity.

We have developed apparatus and methods for identifying miRNAs. We have also developed apparatus and methods for sequencing miRNAs. Moreover, we have developed apparatus and methods for cloning miRNAs.

Before the present apparatus, products and compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

1. Detection

The present invention is related to a microarray comprising a solid substrate comprising a plurality of capture sequences, which may be used for detecting the presence of a target nucleic acid in a sample. A nucleic acid-containing sample may be contacted with the array. Binding of the target nucleic acid to the capture sequence may be detected, and the extent thereof may be measured.

a. Substrate

The solid substrate may be any of the many materials available in the art. Representative examples of solid substrates include glass, plastic or a polymeric substrate.

b. Capture Sequences

(1) First Nucleic Acid

Each capture sequence comprises a first nucleic acid. The first nucleic acid may be a miRNA, a miRNA*, a pre-miRNA, a pri-miRNA, the complement thereof, a nucleic acid substantially identical thereto, or a portion thereof at least 12, 15, 17, 18, 19, 20, 21, 22 or 23 nucleotides, or a DNA encoding said sequence. A substantially identical nucleic acid may have greater than 80%, 85%, 90%, 95%, 97%, 98% or 99% sequence identity to the reference nucleic acid.

Mature miRNAs usually have a length of 19-24 nucleotides, particularly 21, 22 or 23 nucleotides. The miRNAs may also be provided as a precursor which may have a length of 50-90 nucleotides, particularly 60-80 nucleotides. It should be noted that the precursor may be produced by processing of a primary transcript which may have a length of >100 nucleotides.

The nucleic acids may be selected from RNA, DNA or nucleic acid analog molecules, such as sugar- or backbone-modified ribonucleotides or deoxyribonucleotides. It should be noted, however, that other nucleic analogs, such as peptide nucleic acids (PNA) or locked nucleic acids (LNA), are also suitable.

The nucleic acids may be an RNA- or DNA molecule, which contains at least one modified nucleotide analog, i.e. a naturally occurring ribonucleotide or deoxyribonucleotide is substituted by a non-naturally occurring nucleotide. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. The phosphoester group of backbone-modified ribonucleotides connecting to adjacent ribonucleotides may be replaced by a modified group, e.g. of phosphothioate group. It should be noted that the above modifications may be combined.

(2) Second Nucleic Acid

Each capture sequence also comprises a second nucleic acid of at least 20, 25, 30, 35, 40, 45, 50, 55 or 60 nucleotides. The second nucleic acid may be used to anchor the first nucleic acid to the substrate. The second nucleic acid may have features that minimize background hybridization of sample nucleic acids to the capture sequence. For example, the second nucleic acid may not appear in the genome of the organism from which the sample is derived. The second nucleic acid may have also less than 25%, 30%, 35%, 40%,45%, 50%, or 55% identity to any sequence in the genome of the organism from which a sample is derived. Each 10, 11, 12, 13, 14, 15, 16, 17, 18, 19 or 20 nucleotide window of the second nucleic acid may have less than 80% identity to any sequence in the genome of the organism from which a sample is derived. Such properties of the second nucleic acid sequence may yield better specificity compared to the triplet method, which cannot differentiate between binding of a target sequence to the first or second nucleic acid.

(3) Control Sequences

The microarray may comprise one or more negative control sequences. Representative examples of such negative controls include the second nucleic acid sequence by itself, palindrome sequences, mRNA for coding genes, adaptors added in the preparation of the library, tRNA and snoRNA.

The microarray may also comprise mismatch probes. For any given capture sequence, multiple mismatch sequences may be generated by changing nucleotides in different positions of the capture sequence. For example, one or more nucleotide may be replaced with its respective complementary nucleotides (A<->T/U, G<->C, and vice versa). Mismatch control sequences may be used to determine the degree of complementary between the binding between the target sequence and the first nucleic acid. Mismatches in the second nucleic acid may not generate a significant change in the intensity of the probe signal, while mismatches in the first nucleic acid may induce a significant decrease in the probe intensity signal. Mismatches in the first nucleic acid may be used to determine that a particular position does not represent a perfect complementary match between the first nucleic acid and the target sequence.

c. Nucleic Acid Sample

The nucleic acid sample comprises a plurality or library of target sequences. The target sequences may comprise sequences that are substantially complementary to the first nucleic acid. The target sequences may be DNA, RNA or a hybrid thereof.

The target sequences may be prepared by one of many methodologies available in the art. For example, total RNA may be size fractionated to isolate RNA sequences less than or equal to 20, 30, 40, 50, 60, 70, 80, 90, 100, 110, 120, 130, 140, 150, 160, 170, 180, 190 or 200 nucleotides. In one embodiment, the isolated RNA sequences are approximately 20 nucleotides.

Adapters may then be ligated to the 5′- and 3′-ends of the size-fractionated RNA. The 3′ adapter may have a T7 promoter. The 5′- and 3′-adapter may each have restriction sites that allow later cleavage of the adapter. The adapters may be DNA-RNA hybrids. The RNA sequence of the DNA-RNA hybrids may be adjacent to the size-fractionated RNA after ligation.

First strand cDNA may then be produced by reverse transcription. The resulting double-stranded product may then be amplified using the polymerase chain reaction (PCR). PCR may be carried out using labeled nucleotides. The adapters may then be removed from the amplified sequences by using restriction enzymes that are specific for sites present in the design of the adapters. The resulting cDNA products may then be converted to cRNA.

In order to reduce the presence of tRNA sequences in the library, the 3′-adapter may be designed to have a 5′-sequence that yields a restriction site after ligation to the 3′-end of a tRNA. For example, a 5′-adapter sequence of GGT ligated to the 3′-end of a tRNA (ACC at their 3′ end) yields a restriction site for NcoI. Such a restriction enzyme may be used to cleave the tRNA containing sequences prior to or after PCR.

d. Hybridization Analysis

The microarray may be contacted with the nucleic acid sample under stringent or moderately stringent hybridization conditions, thereby allowing a target sequence to hybridize to a sufficiently complementary probe sequence. The intensity at each probe sequence is then measured. The probe signals may be evaluated using parameters including, but not limited to, background signals, controls signals, comparison to signals from mismatch probe sequences.

2. Alternative Detection

The present invention is also related to a method of detecting a target sequence by contacting a plurality of target sequences with a labeled nucleic acid, whereby a labeled nucleic acid may hybridize to a first portion of the target sequence to yield a partial duplex. The partial duplex may then be contacted with a solid substrate comprising a plurality of capture sequences, which may be coupled to color-coded microspheres, whereby a capture sequence may hybridize to a second portion of the target sequence to yield a captured duplex. Binding of the partial duplex to the capture sequence may be detected by measuring the signal of binding at the capture sequence.

3. Sequencing and Cloning

The present invention is also related to a method of sequencing or cloning a target nucleic acid in a sample that hybridizes to a capture sequence coupled to a solid substrate, such as a magnetic bead. In the preparation of the nucleic acid sample, the adapters are not removed from the library of target sequences. The plurality of target sequences comprising 5′- and 3′-adapters is contacted with one or more solid substrates each individually comprising a capture sequence, thereby allowing hybridization of a probe sequence to a target sequence of sufficient complementarity. The bound target sequences may then be dislodged from the solid substrate using any chemical or physical method in the art. The dislodged target sequences may be amplified using primers that hybridize to the 5′- and 3′-adapters. The amplified target sequence may then be cloned or sequenced using any method in the art.

The present invention has multiple aspects, illustrated by the following non-limiting examples.

### EXAMPLE 1

### Design of a Microarray

Microarray chips were produced by attaching various probe sequences of 60 nucleotides to a substrate. The probes contained known or predicted miRNAs, as well as various controls. Known miRNAs were attached to MIRChipl and predicted miRNAs were attached to MIRChip2.

1. Single miRNA Probes

From each miRNA precursor we took a 26-mer containing the miRNA, then assigned 3 probes for each extended miRNA sequence: the 26-mer at the 5′ of the 60-mer probe, the 26-mer at the 3′ of the 60-mer probe, and the 26-mer in the middle of the 60-mer probe. Two different 34-mer sequences which do not appear in the human genome (NHG-sequences) were attached to the 26-mer to complete a 60-mer probe. The NHG-sequences were a combination of 10-mer sequences which are very rare in the human genome. Each potential 34-mer sequence was compared to the human genome by the Blast program and we ended up with 2 different rare NHG-sequences that have an identity of no more than 40% and have no 15-mer sub-sequences with are more than 80% identical.

For a subset of 32 of single miRNA probes we designed an additional 6 mismatch mutation probes: a) a block of 4 mismatches at the 5′ end of the miRNA; b) a block of 6 mismatches at 3′ end of the miRNA; c) one mismatch at position 10 of the miRNA; d) two mismatches at positions 8 and 17 of the miRNA; e) three mismatches at positions 6, 12 and 18 of the miRNA; and f) six mismatches at different positions outside of the miRNA.

2. Duplex miRNA Probes

From each precursor we took a 30-mer containing the miRNA, and then duplicated it to obtain a 60-mer probe. For a subset of 32 of miRNAs we designed additional 3 mismatch mutation probes: a) two mismatches on the first miRNA; b) two mismatches on the second miRNA; and c) two mismatches on each of the miRNAs.

3. Triplex miRNA Probes

Similar to methods described in Krichevsky et al (2003), we attached ˜22 nucleotide long miRNA sequences head-to-tail to obtain 60-mer probes containing up to 3 times the same miRNA sequence. For a subset of 32 probes, we designed an additional 3 mismatch mutation probes: a) two mismatches on the first miRNA; b) two mismatches on the second miRNA; and c) two mismatches on each of the miRNA copies.

4. Precursor with miRNA Probes

For each precursor we took a 60-mer sequence containing the entire miRNA.

5. Precursor without miRNA Probes

For each precursor we took a 60-mer sequence containing no more then 16 nucleotides of the miRNA. For a subset of 32 probes, we designed additional mismatch probes containing 4 mismatches.

6. Controls

General control included the following: 100 probes for mRNAs, representing mostly genes expressed in a wide variety of cell types, 85 representative tRNAs, and 19 representative snoRNA probes. Negative controls included one group composed of 294 randomly chosen 26-mer sequences from the human genome not contained in published precursors sequences, placed at the 5′ and complemented with a 34-mer NHG-sequence. A second group was composed of 182 different 60-mer probes containing different combinations of 10-mer rare sequences.

### EXAMPLE 2

### Design of a Target Library

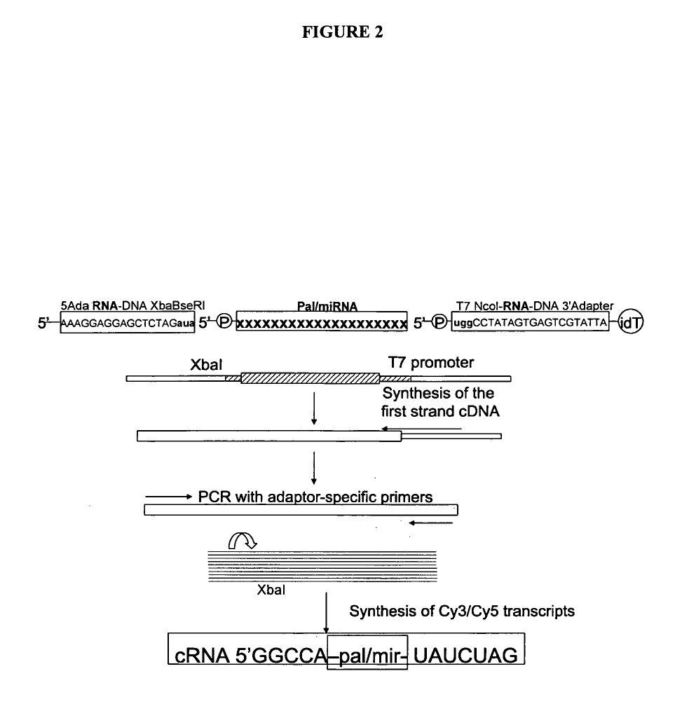

A cDNA target library was made using a procedure similar to that described in Elbashir et al., Genes Dev. 2001 15:188-200. Briefly, total RNA was size-fractionated using an YM-100 column to isolate RNA of about 200 nucleotides. Adaptor sequences were then ligated to the 5′- and 3′-ends of the size-fractionated RNA (FIG. 2). Both adaptors were RNA-DNA hybrids with the RNA portion ligated directly to the size-fractionated total RNA. The 3′-adaptor included a T7 promoter. Either the first or second pair of the following adaptors was used:

TABLE 1

| | |
|---|---|
| 5′ Adapter | |
| 5′-AAAGGAGGAGCTCTAGaua-3′ | SEQ ID NO: 1 |
| 3′ Adapter | |
| 5′-P-uggCCTATAGTGAGTCGTATTA-idT-3′ | SEQ ID NO: 2 |
| 5′ Adapter | |
| 5′-CCTAGGAGGAGGACGTCTGcag-3′ | SEQ ID NO: 3 |
| 3′ Adapter | |
| 5′-P-ccuATAGTGAGTCGTATTATCT-idT-3′ | SEQ ID NO: 4 |

*nucleotides in lowercase represent ribonucleotides and the nucleotides in uppercase represent deoxyribonucleotides

After ligating the adapters to the RNA, the product was converted to first strand cDNA by reverse transcription. The resulting cDNA was then amplified by polymerase chain reaction (PCR) using one of the following pairs of primers:

TABLE 2

| | |
|---|---|
| 5′Primer | |
| 5′-TAATACGACTCACTATAGGCCA-3′ | SEQ ID NO: 5 |
| 3′Primer | |
| 5′-AAAGGAGGAGCTCTAGATA-3′ | SEQ ID NO: 6 |
| 5′Primer | |
| 5′-GCTAGCACTAGTTAATACGACTCACTATAGGCC | SEQ ID NO: 7 |
| A-3′ | |
| 3′Primer | |
| 5′-GCTCTAGGATAATACGACTCACTATAGG-3′ | SEQ ID NO: 8 |
| 5′Primer | |
| 5′-TGACCTGCAGAAAGGAGGAGCTCTAGATA-3′ | SEQ ID NO: 9 |
| 3′Primer | |
| 5′-ATCCTAGGAGGAGGACGTCTGCAG-3′ | SEQ ID NO: 10 |

After amplification, the amplified DNA was digested with Xba1 or Pst to remove the adaptor sequences that were added to the initial RNA. Using the first set of RNA-DNA hybrid adaptors listed above, the first set of primers listed above, and Xba1 yielded cRNA-1. Using the second set of RNA-DNA hybrid adaptors listed above, the second set of primers listed above, and PstI yielded cRNA-2. The resulting cDNA products were then converted to labeled cRNA (1cRNA) incorporating either 3-CTP (Cy3-CTP) or cyanine 5-CTP (Cy5-CTP). The 1cRNA was purified using a G-50 column.

TABLE 3

cRNA Products

| | |
|---|---|
| 5′-GGCCA - pallindrome/miRNA- UAUCUAG-3′ | cRNA-1 |
| 5′-GG- pallindrome/miRNA-C-3′ | cRNA-2 |

### EXAMPLE 3

### Expression Analysis of Known miRNAs

To examine the ability of miRNAs or pre-miRNAs in the 1cRNA to hybridize to theMIRChipl, we examined hybridizations with 5, 17 or 50 μg of 1cRNA derived from HeLa cells. Hybridization solutions that contained the indicated amount of each 1cRNA from either the control or the test samples were prepared using the In situ Hybridization Reagent Kit (Agilent). Hybridized microarrays were scanned using the Agilent LP2 DNA Microarray Scanner at 10 μm resolution. Microarray images were visually inspected for defects.

Microarray images were analyzed using Feature Extraction Software (Version 7.1.1, Agilent). We set the signal of each probe as its median intensity. We observed a nearly constant background intensity signal of 430. Using NHG-sequence negative control probes, the threshold for reliable probe signals was set at 1500. No NHG-sequence probes with signals higher then 1500 were observed in HeLa, Brain, liver and thymus and less then 0.5% of these probes gave signals higher then 1500 in testes and placenta. In all hybridization experiments a high correlation of 0.96 to 0.98 was observed between the Cy5-labeled common control 1cRNA. In addition, 1cRNAs derived from the same RNA source and hybridized to MIRChip1 and MIRChip2 (below) gave a correlation coefficient of 0.98 when identical probes on the two chips were compared.

The hybridization results showed that 17 μg of 1cRNA gave the optimal outcome. In general, signal intensity of miRNA containing probes followed their known abundance in HeLa cells. In contrast, the antisense and 4 palindrome probes outside the miRNA gave no signal above background. This shows that the signals were derived from miRNAs and not from their hairpin precursors.

Of the other controls, signals of tRNA probes were at most similar to those of the most abundant miRNAs, while probes for abundant mRNAs gave only background signals. Hybridizations of MIRChip1 with total RNA oligo-dT derived 1cRNA resulted in the expected pattern of signals from the mRNA probes but no signals above background were observed from the miRNA-containing probes.

### EXAMPLE 4

### Expression Analysis of Predicted miRNAs

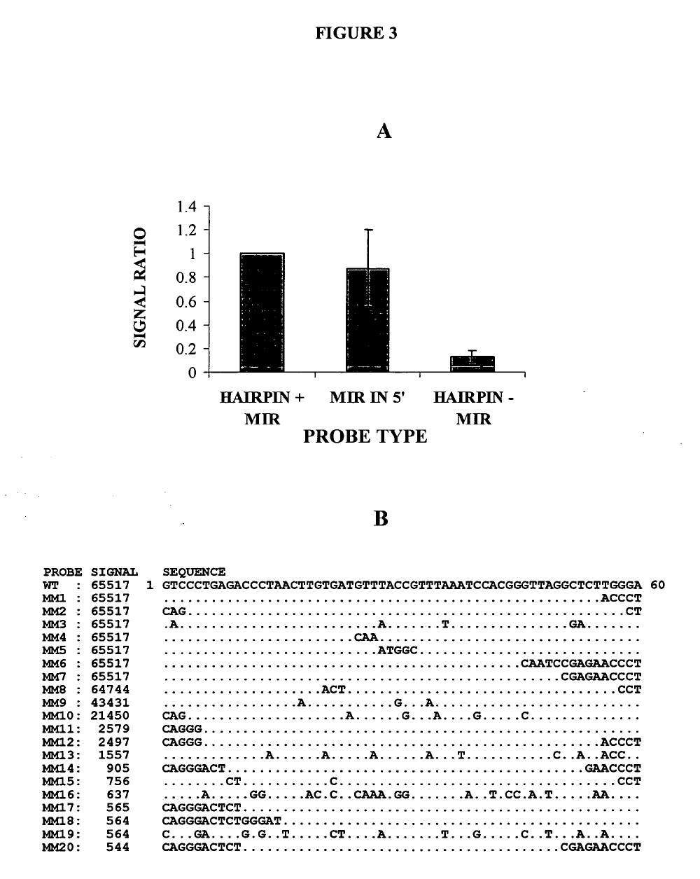

MIRChip2 was hybridized with 17 μg of 1cRNA derived from HeLa cells. A comparison of 60-mer probes containing miRNAs within their precursor sequence to those in which the miRNAs were embedded in NHG-sequences show that both give similar signal levels (FIG. 3A). In contrast, probes containing precursor sequences without miRNAs or with truncated miRNAs gave low or background signals (FIG. 3A). Moreover, a similar hybridization on MIRChip1, which included mismatches either in the miRNA or in the non-miRNA precursor regions, showed that mismatches within the miRNA sequence result in significant reduction in signal intensity while no change is observed in mismatches outside the miRNA (FIG. 3B). Control 60-mer probes composed only of the NHG-sequences gave only background signals. This shows that miRNAs, and not their hairpin precursors, are responsible for the observed signals.

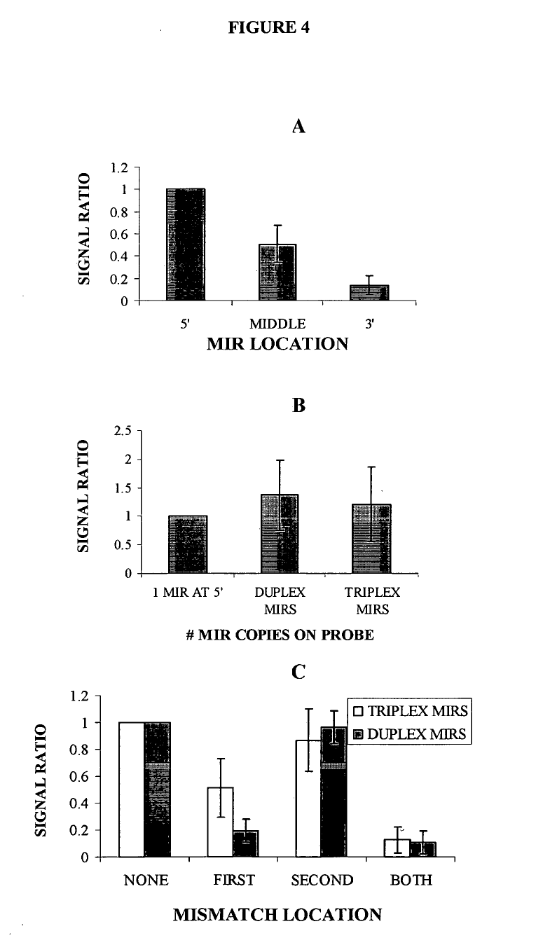

MIRChip2 included miRNAs in three locations along the 60-mer probes to examine the importance of miRNA location. FIG. 4A shows that miRNAs located at the 5′ end of the 60-mer probes result in significantly higher signals then miRNAs located in the middle, with miRNAs located at the 3′ end giving the lowest signals. Comparison of the 60-mer probes containing a single miRNA to the duplex and triplex 60-mer probes show that the inclusion of additional miRNA copies in the 60-mer probes results, at most, in a minor increase in signal intensity (FIG. 4B). Moreover, analysis of duplex and triplex 60-mer probes containing mismatches revealed that, in general, mismatches within the 5′ miRNA cause a significant reduction in signal levels while mismatches in miRNAs located in the middle or in the 3′ ends had a significantly lower effect on signal intensity (FIG. 4C). This shows that a single miRNA located at the 5′ end of the probe, furthest from the surface of the chip, is sufficient to obtain high signals.

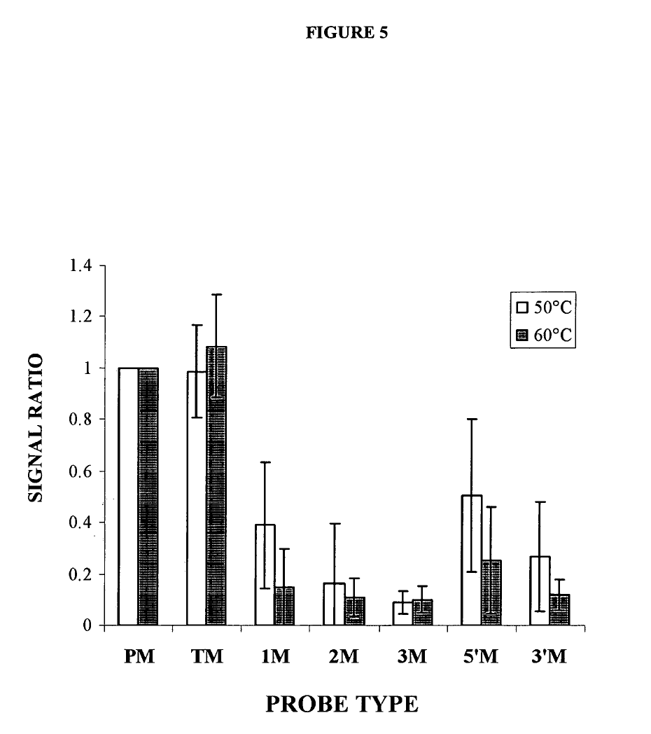

An important control of hybridization specificity is the effect of mismatches on observed signals. FIG. 5 shows the results of hybridizations at temperatures of 50° C. and 60° C. for a subset of 32 miRNAs, each in two different settings of NHG-sequences, for which mismatch-probes were included. While mismatches outside the miRNA sequences did not change signal levels, one, two or three mismatches within the miRNA significantly reduced the signal. In the 60° C. hybridization, even a single mismatch reduced the signal to close to background levels compared to a significantly lower reduction of signal intensity in the 50° C. hybridization, in accordance with the lower stringency of these conditions. Similarly, mismatches in either the 5′ or 3′ regions of the miRNA significantly reduced the signal intensity with higher effects at the 60° C. hybridization temperature. Thus, under the standard conditions using hybridizations at 60° C., specificity was high.

Our findings that single mismatches in the middle of the miRNA sequence, or 2 mismatches in either side, reduce the signal to background levels suggest that the signals are specific. Moreover, miRNAs that are different by few nucleotides from each other often show different expression patterns. As shown below, miRNAs let-7A and let-7B, which differ from each other in only 2 nucleotides, have a very similar pattern, while let-7c, which is one nucleotide different from both let-7A and let-7B, has a different expression pattern with significantly lower expression in placenta and brain but not in the other tissues. Taken together, our data strongly support the specificity of the signals observed using the MIRChip.

### EXAMPLE 5

### Expression Profile of Predicted miRNAs

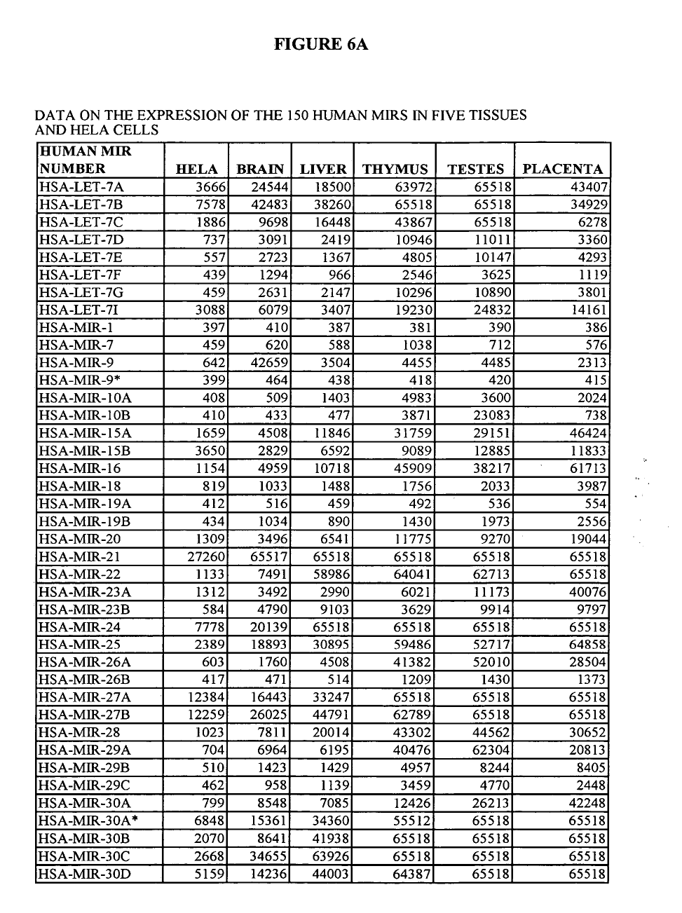

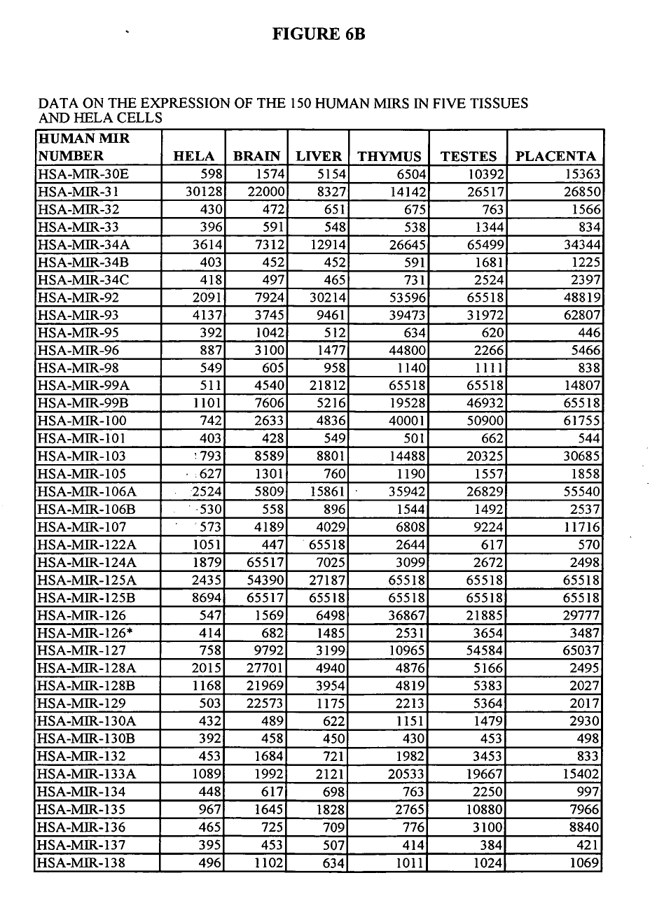

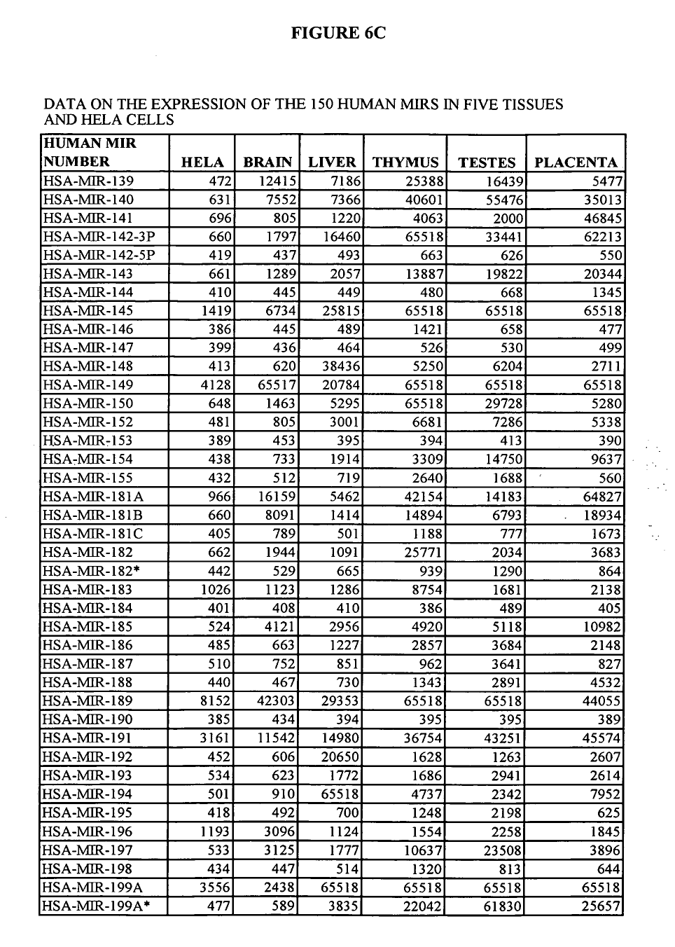

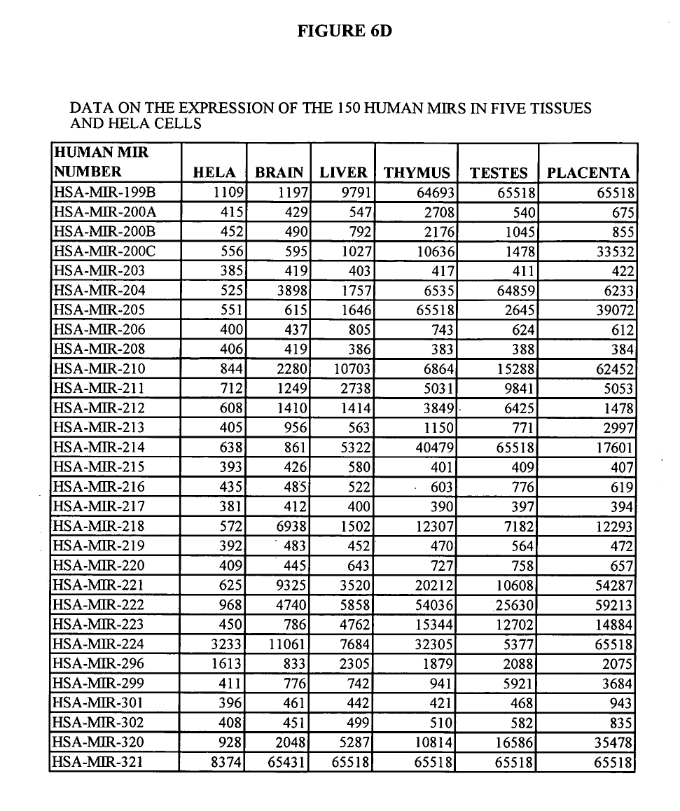

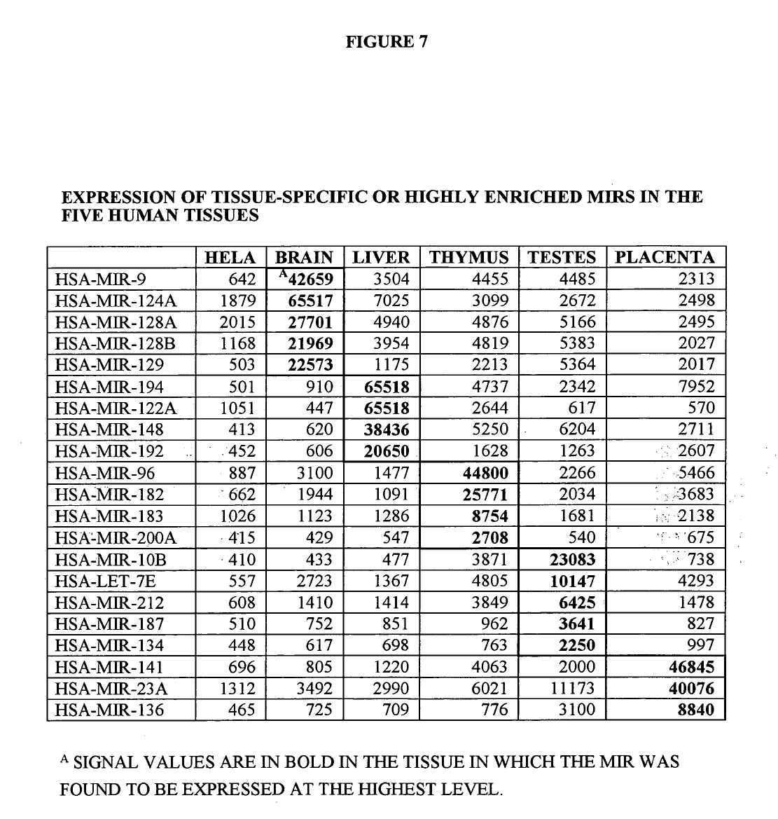

We next hybridized the MIRChip2 with 1cRNAs derived from human brain, liver, thymus, testes, and placenta and examined the tissue specificity of the various miRNAs. The results obtained from the HeLa-cell hybridization mentioned above were included in the analysis. The full set of results can be found in FIG. 6. A comparison was made to results obtained by Sempere et al (2004) that examined the expression of 119 miRNAs by Northern blots in brain and liver as well as other tissues not examined in the present study. Comparison was also made, when relevant, to the oligonucleotide array results of Krichevsky et al (2003) and to the cloning data of Lagos-Quintana et al (2002). MicroRNAs showing distinct brain (e.g. miRNA-9 and miRNA-124A) or liver (miRNA-122A and miRNA-194) tissue specificity gave identical results on our MIRChip hybridizations (FIG. 7). Also, the findings that certain miRNAs, such as let-7A, let-7B, and miRNA-30C (Sempere et al. 2004), are expressed at high levels in many tissues were confirmed using our microarrays, extending the results to the thymus, testes and placenta (FIG. 7). An overall correlation of approximately 0.6 was found between our results and those of Sempere et al. (2004).

We also found distinct differences between our study and those of others. For example, we found very high expression of miRNA-149 in the brain and high expression in the liver whereas Sempere et al. (2004) found low levels in the brain and no signal in the liver. Similarly, we detected significant expression levels of miRNA-20 in both brain and liver compared to no signals on the Northern blots reported by Sempere et al. (2004). On the other hand, miRNA-203 and miRNA-137 showed only background signals in our study compared to high levels of expression in both brain and liver or in the brain, respectively, observed by Sempere et al. (2004).

### EXAMPLE 6

### Further Validation of Expression of Predicted miRNAs

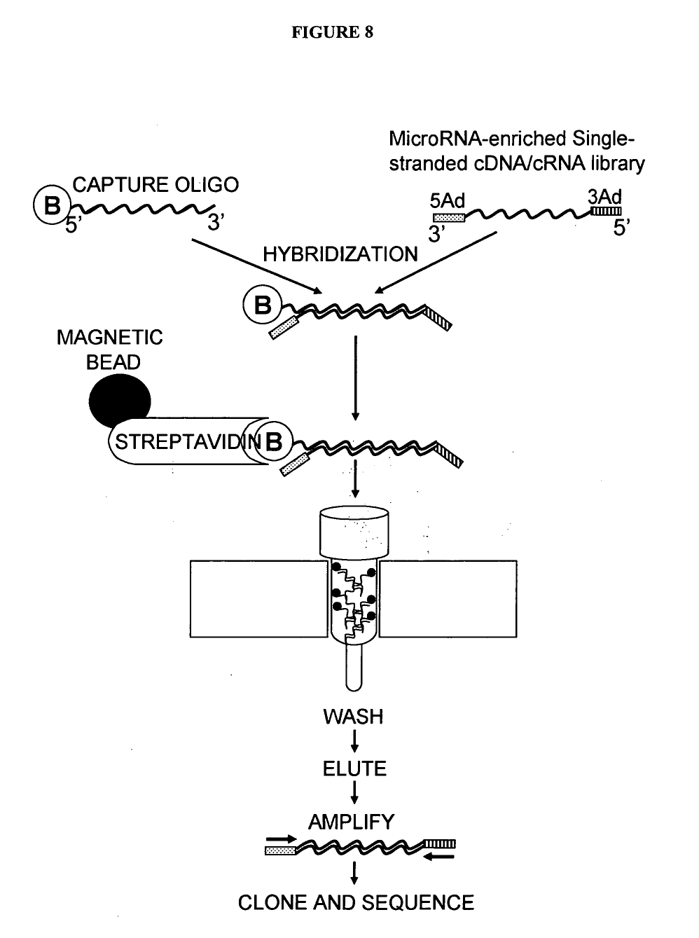

As an additional way of validating expression of the miRNAs, we used a fluorescence-based hybridization method developed by Luminex (Yang et al. 2001) termed “miRNAMASA.” The miRNAMASA technology uses a specific capture-oligo for each targeted miRNA. The capture oligo was covalently coupled onto color-coded microspheres (beads), and was used together with a detection-oligo that was labeled with biotin (FIG. 8). Both capture and detection oligos are spiked with Locked Nucleic Acid (LNA) nucleotides to increase specificity and sensitivity (Petersen and Wengel, 2003). Following hybridization of the capture and detection oligos with the RNA, streptavidine-phycoerythrin is added. The fluorescence associated with the color-coded beads provides a measure for miRNA expression level.

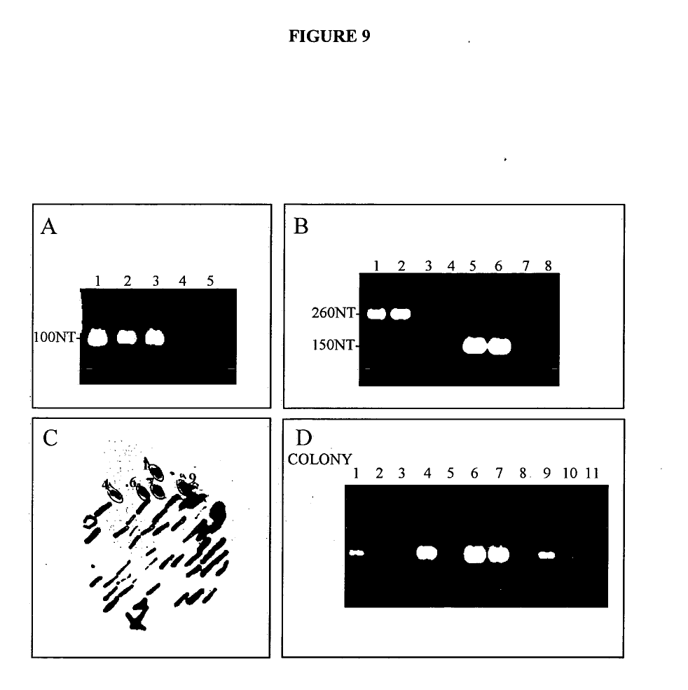

We have focused the miRNAMASA validation study on those miRNAs showing distinct differences between MIRChip1 and the previously published Northern blot data. The analysis was done by multiplexing in two groups. One group included let-7b, miRNA-127, miRNA-129, miRNA-137, miRNA-203 and 5sRNA control. The second group included miRNA-20, miRNA-199a, miRNA-141 and 5sRNA control. The analysis of each group was done on 1 μg of total RNA. A negative bead-control was performed for each group, shown as “blank” in FIG. 9. As shown in FIG. 9, the expression of miRNA-20 was detected in brain and liver, as well in the other three tissues, compared to no signals observed in the Northern blot analysis of Sempere et al. (2004). On the other hand, no expression is observed in any of the tissues for miRNA-137 and miRNA-203 compared to expression of miRNA-137 in the brain and of miRNA-203 in both brain and liver, observed by Sempere et al. (2004). In addition, a good correlation was observed between the miRNAMASA and the MIRChip results in the expression of miRNA-141 and let-7B in all five tissues. These results validate the expression patterns observed in the MIRChip experiments. Examination of the expression of miRNA-127 and miRNA-129 show no signals (FIG. 9) compared to clear expression predicted from the MIRChip experiments. These results are in agreement with the Northern blot data of Sempere et al. (2004).

### EXAMPLE 7

### Clustering Analysis

Clustering analysis was performed on 150 of the miRNAs. For each miRNA, the background signal of 500 was first subtracted from the values observed in all 6 different tissues. A threshold of 30 was set as a minimal value. A log2 transformation was applied, and the Euclidian distance matrix was calculated. A hierarchical clustering using Average Linkage algorithm was performed with an output of a dendrogram. A distance threshold of 6 was used to distinguish between the most significant clusters.

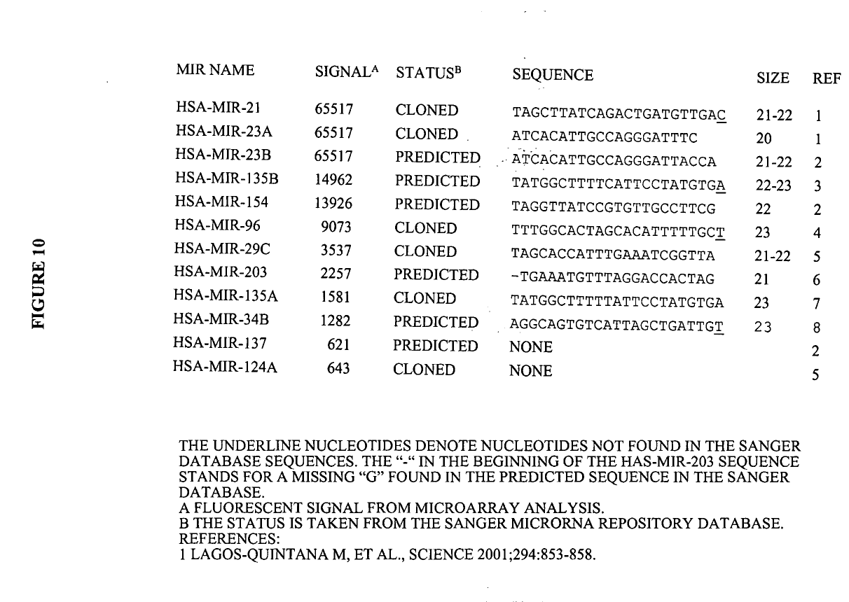

Clustering analysis revealed that miRNAs are expressed in almost every conceivable pattern (FIG. 10). This includes miRNAs expressed in all tissues, miRNAs expressed in some tissues, tissues specific miRNAs and miRNAs undetectable in any of the tissues examined. The analysis revealed distinct clusters of miRNAs specifically expressed in brain, liver, and thymus, while clusters of miRNAs that are specifically expressed in testes and placenta are more obscure. A thorough analysis of the testes, and placenta hybridization data revealed miRNAs that are specific, or highly enriched, in these tissues. FIG. 7 shows, in addition to the brain and liver data, the miRNAs that are tissue-specific or highly enriched in the three tissues not examined before by others: miRNA-96, miRNA-182, and miRNA 183 in the thymus, miRNA-10b, miRNA-212, and miRNA-299 in the testes, and miRNA-141, miRNA-200c, and miRNA-320 in the placenta. Some miRNAs were expressed in two of these tissues (e.g. miRNA-197 and miRNA-205) and others were expressed in all three tissues (e.g. miRNA-26a, miRNA-100, and miRNA-222). Interestingly, we observed an overall low expression of miRNAs in HeLa cells. Only 44 of the miRNAs show signal levels above background compared to 86 to 119 in the five tissues. In addition, none of the miRNAs was found to be specifically enriched in HeLa cells and the vast majority of the miRNAs showing significant signals were expressed at lower levels than in the five tissues. These results are compatible with other reports observing lower expression levels of various miRNAs in cancer cells (Calin et al. 2004, Michael et al. 2003).

### EXAMPLE 8

### Sequence-Directed miRNA Cloning

7. Capture Sequences

Predicted miRNAs were cloned by the MIRAclone method using biotin-labeled capture oligonucleotides which are in reverse-complementary orientation to the library of target molecules. A schematic illustration of the MIRAclone method is presented in FIG. 8. The biotin moiety was added to the 5′ end of the capture sequence.

8. Single-Stranded Library

To construct a library of enriched miRNAs, endogenous 18 to 24 nucleotide RNAs were size-fractionated from total RNA of human placenta tissue. The cDNA preparation procedure was similar to that of Example 2. The 5′- and 3′-adaptors shown below were ligated to the size-fractionated RNA.

TABLE 4

| | |
|---|---|
| 5′ Adapter | |
| 5′-AACTGCAGAAAGGAGGAGCTCTAGata-3′ | SEQ ID NO: 11 |
| 3′ Adapter | |
| 5′-P-uggAACAGATGAATTCTACC-idT-3′ | SEQ ID NO: 12 |

Reverse-transcription was then performed using 3 μg of the adapter-ligated RNA. PCR amplification was then performed using the following primers using an excess of the reverse primer (1:50 ratio) 5′-TAATACGACTCACTATAGGTAGAATTCATCTGTTCCA-3′ (SEQ ID NO: 13). Alternatively, the cRNA was produced by PCR using the same forward primer and a modified reverse primer (5′-ACTGGTGCCTAATACGACTCACTATAGGTAGAAT-3′) (SEQ ID NO: 14) that contained a T7 promoter. This served as a template for in-vitro transcription with T7 RNA polymerase.

9. Hybridization

Hybridization was conducted using 5 μl of the single-stranded PCR products and ˜0.5 μg capture oligonucleotide added to 200 μl TEN buffer (10 mM tris ph=8.0; 1 mM EDTA; 100 mM NaCl). Following hybridization, μMACS Streptavidin Microbeads were added and incubated for 2 minutes at the hybridization temperature. The mixture was then loaded onto a magnetized PMACS Streptavidin Kit columns (130-074-101; Miltenyi Biotec, Gladbach, Germany) and processed according to the manufacturer instructions. The hybridized single-stranded library molecules were eluted by adding 150 μl H₂O pre-heated to 80° C.

10. Sequencing

The recovered single-stranded cDNA library molecules were amplified by PCR using primers for the adaptor sequences. When cRNA was used the PCR was preceded by an RT reaction.

11. Cloning

The recovered single-stranded cDNA library molecules were amplified by PCR using primers for the adaptor sequences. When cRNA was used the PCR was preceded by an RT reaction. PCR products were ligated into a pTZ57/T vector (#k1214, MBI Fermentas, Hanover, Md., USA). The presence of the candidate miRNAs in the ligation products was confirmed by PCR using a primer specific for the candidate miRNAs and a primer located on the 5′ region (FV-primer-5′-CTTCGCTATTACGCCAGCTG-3′) or to the 3′ region (RV-primer-5′-GTTAGCTCACTCATTAGGCACC 3′) of the multiple cloning site of the vector. Positive ligations were transformed into competent JM109 E. coli (L2001, Promega) and plated onto LB-Ampicilin plates with IPTG and Xgal. White and light blue colonies were transferred to duplicate grid-plates, one of which was blotted onto a membrane (Biodyne Plus, Pall) for hybridization with DIG tailed oligonucleotide probes complementary to the expected miRNAs according to manufacturer's instructions (Roche). Positive clones were examined by colony PCR with a miRNAs-specific primer and vector primers as described above. The positive clones were amplified with two external primers on the vector (FV and RV primers). Plasmid DNA from positive colonies was sequenced with a nested primer (5′ GATGTGCTGCAAGGCGATTAAG 3′).

### EXAMPLE 9

### MIRAclone Detection of mir-21

Initially, we tested the MIRAclone method described in Example 8 on human mir-21, which is highly expressed in several tissues (Barad O, 2004). Amplified and non-amplified cDNA resulted in efficient recovery of mir-21. Importantly, no background was observed in the controls, including amplification using the capture oligo itself as template, indicating that the PCR products were derived from the captured and recovered library molecules. Following ligation of the amplified recovered material into the cloning vector we conducted a quality control PCR with mir-21 primer and vector primers to ensure that mir-21 sequences were ligated. As shown in FIG. 9B, mir-21 was present in the ligated vector. Next, the ligation products were transformed into bacteria and positive clones were identified by colony filter hybridization with a mir-21 specific oligonucleotide (FIG. 9C). PCR on colonies, using a mir-21 specific primer and a primer flanking the multiple cloning site in the plasmid, revealed that these clones carried mir-21 sequences (FIG. 9D). Finally, sequencing analysis revealed authentic mir-21 sequences in these clones.

### EXAMPLE 10

### Analysis of MIRAclone Sensitivity

To examine the sensitivity of the MIRAclone method, we tested additional published miRNAs that are expressed at varying levels. As shown in FIG. 10 we were able to obtain clones containing the authentic sequence of all miRNAs that were expressed above background levels in a microarray analysis of placenta-derived miRNAs. This included miRNAs expressed at levels just above the threshold of significant microarray values. Interestingly, MIR-23a and MIR-23b, which differ in only one nucleotide, were each specifically cloned by the respective capture oligo. Thus, the MIRAclone method is highly specific and sensitive, allowing the cloning of miRNAs expressed at relatively low levels.

Several of the published miRNAs were predicted solely by homology to miRNAs in other species, but were never cloned in humans. These include mir-23b, mir-34b, mir-135b, mir-154, and mir-203 (FIG. 10). Thus, the MIRAclone method was able to provide the first evidence for their expression in human cells. In addition, for mir-21, mir-34b, mir-96, mir-135b, and mir-203 we found variations from the published sequences (FIG. 10). While for the first four miRNAs the variations were in the 3′ end, which is commonly observed among many miRNAs, the cloned mir-203 sequence lacked the first 5′ “G” found in the predicted sequence.

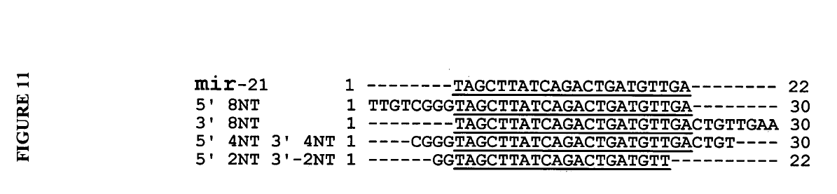

An additional feature we have examined is the flexibility and sensitivity of the method when longer capturing oligonucleotides are used. This is important since prediction algorithms cannot always predict the precise location of the mature miRNA within the hairpin precursor. We created variations of the mir-21 capture oligonucleotides with 8 additional nucleotides from the precursor sequence on the 5′ side of mir-21 (5′ 8 nt), 8 additional nucleotides from the precursor sequence on the 3′ side of mir-21 (3′ 8 nt), and 4 nucleotides on the 5′ and 4 nucleotides on the 3′ side of mir-21 (5′ 4 nt 3′ 4 nt) (FIG. 11). In addition, the sequence of the capture oligonucleotide was shifted by 2 nucleotides, leaving only 20 nucleotides that match the mature miRNA (FIG. 11). We found that the cloning efficiency of all three longer capture oligo was comparable to that of the parental capture oligo. In addition, the reduction of match-length of mir-21 capture oligo by two nucleotides did not change the cloning efficiency.

### EXAMPLE 11

### Cloning of Computationally Predicted MicroRNAs

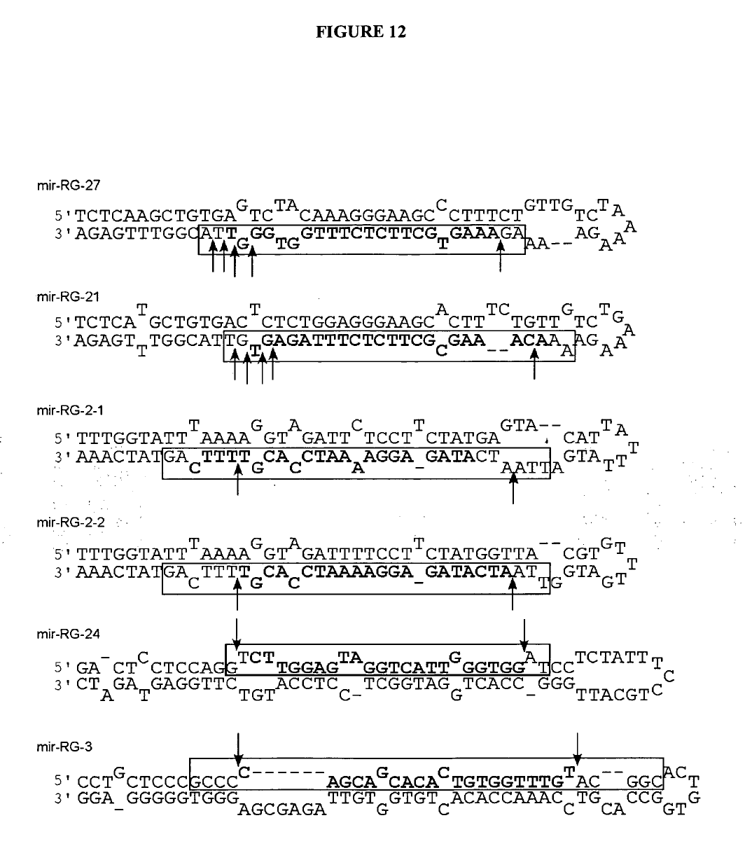

We selected for cloning 55 miRNAs predicted using methods similar to those described in U.S. Patent Application No. 60/522,459, Ser. Nos. 10/709,577 and 10/709,572. The design of the 26-30 nucleotide long capture oligonucleotides was based on the prediction of miRNA location within the predicted hairpin precursors. 2-4 nucleotides were added on each side of the 22-mer predicted miRNA. As described above, these additional nucleotides do not impede miRNA detection. The capture oligo designed for mir-RG-2 is identical to both mir-RG-2-1 and mir-RG-2-2 precursors as these two precursors share an identical 3′ stem (FIG. 12). Interestingly, due to differences in the 5′ stem sequence the miRNAs predicted from these two precursors vary slightly in position along the 3′ stem. In addition, it should be noted that these two precursors are found within 700 bp of each other on chromosome 19.

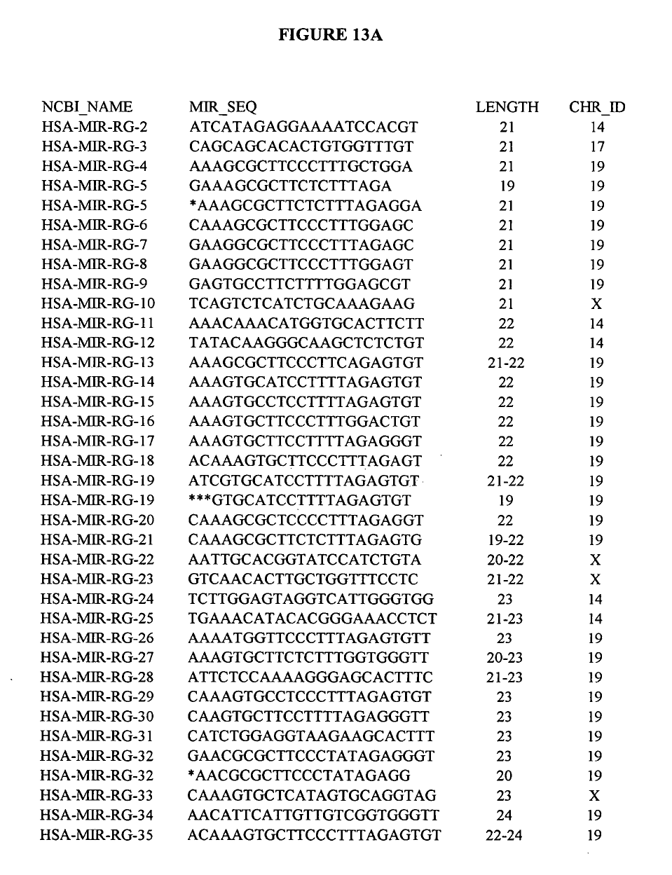

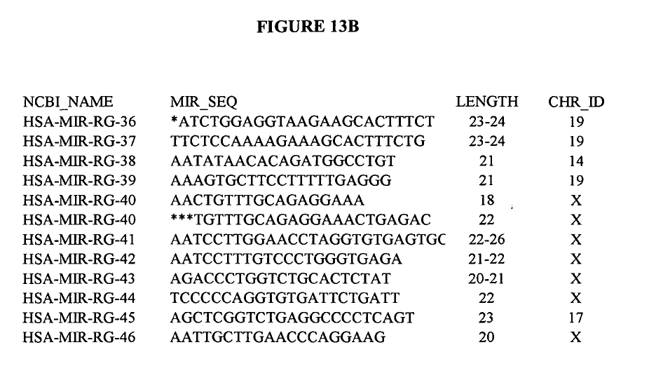

Using the designed capture oligonucleotides we successfully cloned 45 novel miRNAs, including all five predicted miRNAs shown on FIG. 12. FIG. 13 presents the full set of cloned miRNAs. The 45 novel miRNAs were derived from 35 capture oligonucleotides as clones derived from some of the capture oligonucleotides resulted in the cloning of two or more miRNAs of related sequence. The expression of the five miRNAs shown in FIG. 12 was further validated by Northern blot analysis, which showed three of the miRNAs (mir-RG-27, mir-RG-21, and mir-RG-2) are expressed predominantly in placenta, mir-RG-24 is expressed both in placenta and brain, and mir-RG-3 is expressed in the brain, liver and thymus.

Many of the cloned miRNAs have shown 3′ sequence length heterogeneity as found in previous cloning studies. However, for four miRNAs we have also found heterogeneity in the 5′ end. Two miRNAs, mir-RG-31 and mir-RG-36, can also be regarded as the same miRNA showing 5′ sequence length heterogeneity; however, mir-RG-31 is uniquely encoded by another hairpin precursor and thus regarded as a unique miRNA. Two of the cases showing apparent 3′ sequence heterogeneity may actually be interpreted as two different miRNAs processed from two different precursors. Thus, the 22 nucleotide long mir-RG-18 can be processed from two different precursors while mir-RG-35, which have the same 22 nucleotides but the later is 2 nucleotides longer, is encoded by only one of these palindromes. Similarly, mir-RG-31 and mir-RG-36, which differ in both 3′ and 5′ sequences, share the same precursor though mir-RG-31 is found also on another precursor.

In three cases we have found two miRNAs encoded by the different stems of the precursor. This includes mir-RG-9 and mir-RG-37, mir-RG-10 and mir-RG-40, and mir-RG-15 and mir-RG-28. Thus, one the miRNAs in these pairs can be regarded as miRNAs*. In four cases we have found a miRNAs that matches two precursors. This includes the mir-RG-9 and mir-RG-37 pair that are encoded by two identical precursors, as well as mir-RG-2, mir-RG-4, and mir-RG-14.

For some of the cloned miRNAs a match between the predicted and cloned sequences was observed, exemplified by mir-RG-27 and mir-RG-2-2 in FIG. 12. For other miRNAs a minimal difference of 1 nucleotide in the 5′ (e.g. mir-RG-21 and mir-RG-3 in FIG. 12) or both 5′ and 3′ (e.g. mir-RG-24 in FIG. 12) was observed. Thus, in general, our miRNA-prediction algorithm seems accurate.

1. A method of detecting a miRNA comprising
(a) providing an array comprising a solid substrate and a plurality of positionally distinguishable polynucleotides attached to the solid substrate, wherein each polynucleotide comprises a miRNA;
(b) contacting the array of (a) with a plurality of target polynucleotides comprising a complement of a miRNA under conditions permitting hybridization; and
(c) detecting hybridization of a target sequence to the miRNA of (a), wherein a miRNA is detected when the hybridization of (c) is above background.

2. The method of claim 1 wherein the plurality of target polynucleotides is produced by a method comprising:
(a) providing RNA comprising a plurality of miRNA;
(b) ligating adapters to the 5′ and 3′ ends of the miRNA;
(c) preparing first strand cDNA of the 5′-adapter-miRNA-adapter-3′;
(d) amplifying the adapter-miRNA-adapter; and
(e) preparing cRNA using a promoter complementary to the 3′ adapter.

3. The method of claim 2 wherein the RNA is less than 160 nucleotides in length.

4. The method of claim 2 wherein the adapters are DNA-RNA hybrids.

5. The method of claim 2 wherein the adapters comprise a restriction site.

6. The method of claim 5 wherein either prior to or after amplification, the adapters are removed by restriction digestion.

7. A method of detecting a miRNA, comprising:
(a) providing a plurality of target polynucleotides comprising a miRNA;
(b) providing a labeled oligo that is complementary to a portion of the target nucleotides;
(c) providing a substrate comprising a capture oligonucleotide comprising at least 16 nucleotides of a miRNA complementary sequence;
(d) contacting the target nucleotides, labeled oligo and substrate; and
(e) detecting hybridization of the target nucleotides, labeled oligo and substrate.

8. A method of isolating a miRNA comprising:
(a) providing a solid substrate comprising a capture oligonucleotide comprising at least 16 nucleotides of a miRNA sequence;
(b) contacting the capture oligonucleotide with a plurality of target polynucleotides comprising a complement of a miRNA under conditions permitting hybridization; and
(c) eluting the target polynucleotides from the capture oligonucleotide.

9. The method of sequencing an miRNA, comprising sequencing a miRNA isolated by the method of claim 8;

10. The method of cloning an miRNA, comprising cloning a miRNA isolated by the method of claim 8;


SEQUENCE LISTING (108 sequences)

SEQ ID NO: 1 - DNA, 19 bp, Artificial sequence
  Exemplary genomic sequence
aaaggaggagctctagaua

SEQ ID NO: 2 - DNA, 22 bp, Artificial sequence
  Exemplary genomic sequence
uggcctatagtgagtcgtatta

SEQ ID NO: 3 - DNA, 22 bp, Artificial sequence
  Exemplary genomic sequence
cctaggaggaggacgtctgcag

SEQ ID NO: 4 - DNA, 22 bp, Artificial sequence
  Exemplary genomic sequence
ccuatagtgagtcgtattatct

SEQ ID NO: 5 - DNA, 22 bp, Artificial sequence
  Exemplary genomic sequence
taatacgactcactataggcca

SEQ ID NO: 6 - DNA, 19 bp, Artificial sequence
  Exemplary genomic sequence
aaaggaggagctctagata

SEQ ID NO: 7 - DNA, 34 bp, Artificial sequence
  Exemplary genomic sequence
gctagcactagttaatacgactcactataggcca

SEQ ID NO: 8 - DNA, 28 bp, Artificial sequence
  Exemplary genomic sequence
gctctaggataatacgactcactatagg

SEQ ID NO: 9 - DNA, 29 bp, Artificial sequence
  Exemplary genomic sequence
tgacctgcagaaaggaggagctctagata

SEQ ID NO: 10 - DNA, 24 bp, Artificial sequence
  Exemplary genomic sequence
atcctaggaggaggacgtctgcag

SEQ ID NO: 11 - DNA, 27 bp, Artificial sequence
  Exemplary genomic sequence
aactgcagaaaggaggagctctagata

SEQ ID NO: 12 - DNA, 20 bp, Artificial sequence
  Exemplary genomic sequence
uggaacagatgaattctacc

SEQ ID NO: 13 - DNA, 37 bp, Artificial sequence
  Exemplary genomic sequence
taatacgactcactataggtagaattcatctgttcca

SEQ ID NO: 14 - DNA, 34 bp, Artificial sequence
  Exemplary genomic sequence
actggtgcctaatacgactcactataggtagaat

SEQ ID NO: 15 - DNA, 20 bp, Artificial sequence
  Exemplary genomic sequence
cttcgctattacgccagctg

SEQ ID NO: 16 - DNA, 22 bp, Artificial sequence
  Exemplary genomic sequence
gttagctcactcattaggcacc

SEQ ID NO: 17 - DNA, 22 bp, Artificial sequence
  Exemplary genomic sequence
gatgtgctgcaaggcgattaag

SEQ ID NO: 18 - DNA, 60 bp, Artificial sequence
  Synthetic
gtccctgagaccctaacttgtgatgtttaccgtttaaatccacgggttaggctcttggga

SEQ ID NO: 19 - DNA, 60 bp, Artificial sequence
  Synthetic
gtccctgagaccctaacttgtgatgtttaccgtttaaatccacgggttaggctctaccct

SEQ ID NO: 20 - DNA, 60 bp, Artificial sequence
  Synthetic
cagcctgagaccctaacttgtgatgtttaccgtttaaatccacgggttaggctcttggct

SEQ ID NO: 21 - DNA, 60 bp, Artificial sequence
  Synthetic
gaccctgagaccctaacttgtgatgttaaccgttttaatccacgggttagggacttggga

SEQ ID NO: 22 - DNA, 60 bp, Artificial sequence
  Synthetic
gtccctgagaccctaacttgtgatcaataccgtttaaatccacgggttaggctcttggga

SEQ ID NO: 23 - DNA, 60 bp, Artificial sequence
  Synthetic
gtccctgagaccctaacttgtgatgttatggctttaaatccacgggttaggctcttggga

SEQ ID NO: 24 - DNA, 60 bp, Artificial sequence
  Synthetic
gtccctgagaccctaacttgtgatgtttaccgtttaaatccacggcaatccgagaaccct

SEQ ID NO: 25 - DNA, 60 bp, Artificial sequence
  Synthetic
gtccctgagaccctaacttgtgatgtttaccgtttaaatccacgggttagcgagaaccct

SEQ ID NO: 26 - DNA, 60 bp, Artificial sequence
  Synthetic
gtccctgagaccctaacttgtgatgtttaccgtttaaatccacgggttaggctcttgcct

SEQ ID NO: 27 - DNA, 60 bp, Artificial sequence
  Synthetic
gtccctgagaccctaacatgtgatgtttagcgtataaatccacgggttaggctcttggga

SEQ ID NO: 28 - DNA, 60 bp, Artificial sequence
  Synthetic
cagcctgagaccctaacttgtgaagtttacggttaaaatgcacggcttaggctcttggga

SEQ ID NO: 29 - DNA, 60 bp, Artificial sequence
  Synthetic
cagggtgagaccctaacttgtgatgtttaccgtttaaatccacgggttaggctcttggga

SEQ ID NO: 30 - DNA, 60 bp, Artificial sequence
  Synthetic
cagggtgagaccctaacttgtgatgtttaccgtttaaatccacgggttaggctctaccct

SEQ ID NO: 31 - DNA, 60 bp, Artificial sequence
  Synthetic
gtccctgagacccaaacttgagatgtataccgtataattccacgggttacgcactaccga

SEQ ID NO: 32 - DNA, 60 bp, Artificial sequence
  Synthetic
cagggactgaccctaacttgtgatgtttaccgtttaaatccacgggttaggctgaaccct

SEQ ID NO: 33 - DNA, 60 bp, Artificial sequence
  Synthetic
gtccctgactccctaacttgtcatgtttaccgtttaaatccacgggttaggctcttgcct

SEQ ID NO: 34 - DNA, 60 bp, Artificial sequence
  Synthetic
gtcccagagacggtaactactcatcaaaagggtttaaaacctcccgattggctcaaggga

SEQ ID NO: 35 - DNA, 60 bp, Artificial sequence
  Synthetic
cagggactctccctaacttgtgatgtttaccgtttaaatccacgggttaggctcttggga

SEQ ID NO: 36 - DNA, 60 bp, Artificial sequence
  Synthetic
cagggactctgggatacttgtgatgtttaccgtttaaatccacgggttaggctcttggga

SEQ ID NO: 37 - DNA, 60 bp, Artificial sequence
  Synthetic
ctccgagagagcgtatcttgtcttgttaaccgttttaatgcacggctttggcactaggga

SEQ ID NO: 38 - DNA, 60 bp, Artificial sequence
  Synthetic
cagggactctccctaacttgtgatgtttaccgtttaaatccacgggttagcgagaaccct

SEQ ID NO: 39 - DNA, 23 bp, Human
tagcttatcagactgatgttgac

SEQ ID NO: 40 - DNA, 20 bp, Human
atcacattgccagggatttc

SEQ ID NO: 41 - DNA, 22 bp, Human
atcacattgccagggattacca

SEQ ID NO: 42 - DNA, 23 bp, Human
tatggcttttcattcctatgtga

SEQ ID NO: 43 - DNA, 22 bp, Human
taggttatccgtgttgccttcg

SEQ ID NO: 44 - DNA, 23 bp, Human
tttggcactagcacatttttgct

SEQ ID NO: 45 - DNA, 22 bp, Human
tagcaccatttgaaatcggtta

SEQ ID NO: 46 - DNA, 21 bp, Human
tgaaatgtttaggaccactag

SEQ ID NO: 47 - DNA, 23 bp, Human
tatggctttttattcctatgtga

SEQ ID NO: 48 - DNA, 23 bp, Human
aggcagtgtcattagctgattgt

SEQ ID NO: 49 - DNA, 22 bp, Human
tagcttatcagactgatgttga

SEQ ID NO: 50 - DNA, 30 bp, Artificial sequence
  Synthetic
ttgtcgggtagcttatcagactgatgttga

SEQ ID NO: 51 - DNA, 30 bp, Artificial sequence
  Synthetic
tagcttatcagactgatgttgactgttgaa

SEQ ID NO: 52 - DNA, 30 bp, Artificial sequence
  Synthetic
cgggtagcttatcagactgatgttgactgt

SEQ ID NO: 53 - DNA, 22 bp, Artificial sequence
  Synthetic
ggtagcttatcagactgatgtt

SEQ ID NO: 54 - DNA, 87 bp, human
tctcaagctgtgagtctacaaagggaagccctttctgttgtctaaaagaaaagaaagtgcttctctttggtgggttacggtttgaga

SEQ ID NO: 55 - DNA, 87 bp, human
tctcatgctgtgactctctggagggaagcactttctgttgtctgaaagaaaacaaagcgcttctctttagagtgttacggtttgaga

SEQ ID NO: 56 - DNA, 86 bp, Human
tttggtatttaaaaggtagattctccttctatgagtacattatttatgattaatcatagaggaaaatccacgttttcagtatcaaa

SEQ ID NO: 57 - DNA, 86 bp, Human
tttggtatttaaaaggtagattttccttctatggttacgtgtttgatggttaatcatagaggaaaatccacgttttcagtatcaaa

SEQ ID NO: 58 - DNA, 92 bp, Human
gactcctccaggtcttggagtaggtcattgggtggatcctctatttcctgcattgggccactggatggctcctccatgtcttggagtagatc

SEQ ID NO: 59 - DNA, 91 bp, Human
cctgctcccgccccagcagcacactgtggtttgtacggcactgtggccacgtccaaaccacactgtggtgttagagcgagggtgggggagg

SEQ ID NO: 60 - DNA, 21 bp, Human
atcatagaggaaaatccacgt

SEQ ID NO: 61 - DNA, 21 bp, Human
cagcagcacactgtggtttgt

SEQ ID NO: 62 - DNA, 21 bp, Human
aaagcgcttccctttgctgga

SEQ ID NO: 63 - DNA, 19 bp, Human
gaaagcgcttctctttaga

SEQ ID NO: 64 - DNA, 21 bp, Human
aaagcgcttctctttagagga

SEQ ID NO: 65 - DNA, 21 bp, Human
caaagcgcttccctttggagc

SEQ ID NO: 66 - DNA, 21 bp, Human
gaaggcgcttccctttagagc

SEQ ID NO: 67 - DNA, 21 bp, Human
gaaggcgcttccctttggagt

SEQ ID NO: 68 - DNA, 21 bp, Human
gagtgccttcttttggagcgt

SEQ ID NO: 69 - DNA, 21 bp, Human
tcagtctcatctgcaaagaag

SEQ ID NO: 70 - DNA, 22 bp, Human
aaacaaacatggtgcacttctt

SEQ ID NO: 71 - DNA, 22 bp, Human
tatacaagggcaagctctctgt

SEQ ID NO: 72 - DNA, 22 bp, Human
aaagcgcttcccttcagagtgt

SEQ ID NO: 73 - DNA, 22 bp, Human
aaagtgcatccttttagagtgt

SEQ ID NO: 74 - DNA, 22 bp, Human
aaagtgcctccttttagagtgt

SEQ ID NO: 75 - DNA, 22 bp, Human
aaagtgcttccctttggactgt

SEQ ID NO: 76 - DNA, 22 bp, Human
aaagtgcttccttttagagggt

SEQ ID NO: 77 - DNA, 22 bp, Human
acaaagtgcttccctttagagt

SEQ ID NO: 78 - DNA, 22 bp, Human
atcgtgcatccttttagagtgt

SEQ ID NO: 79 - DNA, 19 bp, Human
gtgcatccttttagagtgt

SEQ ID NO: 80 - DNA, 22 bp, Human
caaagcgctcccctttagaggt

SEQ ID NO: 81 - DNA, 22 bp, Human
caaagcgcttctctttagagtg

SEQ ID NO: 82 - DNA, 22 bp, Human
aattgcacggtatccatctgta

SEQ ID NO: 83 - DNA, 22 bp, Human
gtcaacacttgctggtttcctc

SEQ ID NO: 84 - DNA, 23 bp, Human
tcttggagtaggtcattgggtgg

SEQ ID NO: 85 - DNA, 23 bp, Human
tgaaacatacacgggaaacctct

SEQ ID NO: 86 - DNA, 23 bp, Human
aaaatggttccctttagagtgtt

SEQ ID NO: 87 - DNA, 23 bp, Human
aaagtgcttctctttggtgggtt

SEQ ID NO: 88 - DNA, 23 bp, Human
attctccaaaagggagcactttc

SEQ ID NO: 89 - DNA, 23 bp, Human
caaagtgcctccctttagagtgt

SEQ ID NO: 90 - DNA, 23 bp, Human
caagtgcttccttttagagggtt

SEQ ID NO: 91 - DNA, 23 bp, Human
catctggaggtaagaagcacttt

SEQ ID NO: 92 - DNA, 23 bp, Human
gaacgcgcttccctatagagggt

SEQ ID NO: 93 - DNA, 20 bp, Human
aacgcgcttccctatagagg

SEQ ID NO: 94 - DNA, 23 bp, Human
caaagtgctcatagtgcaggtag

SEQ ID NO: 95 - DNA, 24 bp, Human
aacattcattgttgtcggtgggtt

SEQ ID NO: 96 - DNA, 24 bp, Human
acaaagtgcttccctttagagtgt

SEQ ID NO: 97 - DNA, 24 bp, Human
atctggaggtaagaagcactttct

SEQ ID NO: 98 - DNA, 24 bp, Human
ttctccaaaagaaagcactttctg

SEQ ID NO: 99 - DNA, 21 bp, Human
aatataacacagatggcctgt

SEQ ID NO: 100 - DNA, 21 bp, Human
aaagtgcttcctttttgaggg

SEQ ID NO: 101 - DNA, 18 bp, Human
aactgtttgcagaggaaa

SEQ ID NO: 102 - DNA, 22 bp, Human
tgtttgcagaggaaactgagac

SEQ ID NO: 103 - DNA, 26 bp, Human
aatccttggaacctaggtgtgagtgc

SEQ ID NO: 104 - DNA, 22 bp, Human
aatcctttgtccctgggtgaga

SEQ ID NO: 105 - DNA, 21 bp, Human
agaccctggtctgcactctat

SEQ ID NO: 106 - DNA, 22 bp, Human
tcccccaggtgtgattctgatt

SEQ ID NO: 107 - DNA, 23 bp, Human
agctcggtctgaggcccctcagt

SEQ ID NO: 108 - DNA, 20 bp, Human
aattgcttgaacccaggaag